(19) United States
(12) Patent Application Publication
(10) Pub. No.: US 2010/0070019 A1
(43) Pub. Date: Mar. 18, 2010

(54) EXTRA-VASCULAR WRAPPING FOR TREATING ANEURYSMATIC AORTA AND METHODS THEREOF

(75) Inventor: Alon Shalev, Ra 'anana (IL)

Correspondence Address: Fleit Gibbons Gutman Bongini & Bianco PL, 21355 EAST DIXIE HIGHWAY, SUITE 115, MIAMI, FL, 33180, US

(73) Assignee: ANEUWRAP LTD., RA'ANANA (IL)

(21) Appl. No.: 12/447,684

(22) PCT Filed: Oct. 29, 2007
(86) PCT No.: PCT/IL07/01312

§ 371 Date: Apr. 29, 2009

Related U.S. Application Data
(60) Provisional application No. 60/863,373, filed on Oct. 29, 2006.

(51) Int. Cl. A61F 2/82

(52) U.S. Cl. 623/1.15; 623/1.46

(57) ABSTRACT

A new extra-vascular wrapping (EVW) is disclosed. The EVW comprises of a (i) wrapping adapted to at least partially encircle at least a segment of an aorta, especially an aneurysmatic aorta, and (ii) anchoring means located at the distal portion of the wrapping adapted to immobilize the wrapping to the aorto-iliac bifurcation via engagement of the aorto-iliac junction, wherein the bifurcation-anchored wrapping is applied external to the aorta and further wherein the wrapping is collapse-resistant in the proximal-to-distal direction of the aorta. The EVW is presented with its various embodiments, such as gusset-like structured EVW, Sustained drug delivery EVW, supported EVW and ringed EVW. Methods for treating aneurysmatic aorta in an open-surgery procedure by utilizing the EVWs and by applying step of positioning a macroporous medical textile externally adjacent to the aorta are also disclosed.

### FIELD OF THE INVENTION

The present invention generally pertains to an extra-vascular wrapping immobilized to the aorto-iliac bifurcation via engagement of the aorto-iliac junction for treating abdominal aneurysmatic aorta, and methods thereof.

### BACKGROUND OF THE INVENTION

An aneurysm is a localized, blood-filled dilation (bulge) of a blood vessel caused by disease or weakening of the vessel wall. Left untreated, the aneurysm will frequently rupture, resulting in loss of blood through the rupture and death.

Aneurysms may involve arteries or veins and have various causes. They are commonly further classified by shape, structure and location. Aortic aneurysms are the most common form of arterial aneurysm and are life threatening. The aorta is the main artery which supplies blood to the circulatory system. The aorta arises from the left ventricle of the heart, passes upward and bends over behind the heart, and passes down through the thorax and abdomen. Among other arterial vessels branching off the aorta along its path, the abdominal aorta supplies two side vessels to the kidneys, the renal arteries. Below the level of the renal arteries, the abdominal aorta continues to about the level of the fourth lumbar vertebrae (or the navel), where it divides into the iliac arteries. The iliac arteries, in turn, supply blood to the lower extremities and perineal region.

It is common for an aortic aneurysm to occur in that portion of the abdominal aorta between the renal arteries and the iliac arteries. This portion of the abdominal aorta is particularly susceptible to weakening, resulting in an aortic aneurysm. Such an aneurysm is often located near the iliac arteries. An aortic aneurysm larger than about 5 cm in diameter in this section of the aorta is ominous. Left untreated, the aneurysm may rupture, resulting in rapid, and usually fatal, hemorrhaging. Typically, a surgical procedure is not performed on aneurysms smaller than 5 cm because no statistically demonstrated benefit exists in performing such procedures.

Aneurysms in the abdominal aorta are associated with a particularly high mortality rate; accordingly, current medical standards call for urgent operative repair when aneurysm diameter is larger than 5 cm. Abdominal surgery, however, results in substantial stress to the body. Although the mortality rate for an aortic aneurysm is extremely high, there is also considerable mortality and morbidity associated with open surgical intervention to repair an aortic aneurysm.

Repair of an aortic aneurysm by surgical means is a major operative procedure. Substantial morbidity accompanies the procedure, resulting in a protracted recovery period. Further, the procedure entails a substantial risk of morbidity and mortality, mostly due to the cardiopulmonary bypass employed in such a procedure.

Therefore, less invasive methods have been introduced to attempt to treat an aortic aneurysm without the attendant risks of intra-abdominal surgery. Among them are inventions such as U.S. Pat. No. 4,562,596 which discloses an aortic graft constructed for intraluminal insertion; U.S. Pat. No. 4,787,899 teaches an intraluminal grafting system includes a hollow graft which has a plurality of staples adapted proximate its proximal end. The system includes a guide for positioning the proximal end of the graft upstream in a lumen which may be a blood vessel or artery; and U.S. Pat. No. 5,042,707 which presents a stapler, adapted to be inserted into a blood vessel and moved to a desired position therealong, and having a selectively-articulatable distal marginal end portion used to staple a graft to the interior wall of the blood vessel.

Hence, although in recent years certain techniques have been developed that may reduce the stress, morbidity, and risk of mortality associated with surgical intervention to repair aortic aneurysms, none of the systems that have been developed effectively treat the aneurysm and exclude the affected section of aorta from the pressures and stresses associated with circulation. None of the devices disclosed in the references provide a reliable and quick means to reinforce an aneurysmal artery. In addition, all of the prior references require a sufficiently large section of healthy aorta surrounding the aneurysm to ensure attachment of the graft. The neck of the aorta at the cephalad end (i.e., above the aneurysm) is usually sufficient to maintain a graft's attachment means. However, when an aneurysm is located near the iliac arteries, there may be an ill-defined neck or no neck below the aneurysm. Such an ill-defined neck would have an insufficient amount of healthy aortic tissue to which to successfully mount a graft. Furthermore, much of the abdominal aorta wall may be calcified which may make it extremely difficult to attach the graft to the wall.

Shortcomings of the presently available endovascular stent-graft products include endoleaks, anatomic variability, anatomic non-conformity, migration/dislocation, discontinuities of endoluminal profile and hrombogenicity.

Endoleaks are caused by passage of blood into the aneurismal space subsequent to stent-graft placement. Research has exposed yet another problem which indicates that the necks of the post-surgical aorta increase in size for approximately twelve months, regardless of whether the aneurysm experiences dimensional change. This phenomenon can result in perigraft leaks and graft migration. Anatomic variability: although sizing of “tube” or “bifurcated” grafts is radiographically assessed prior to surgery, it is necessary for the surgeon to have a large selection of graft lengths and diameters on hand to ensure an appropriate surgical outcome. Anatomic non-conformity: placement of a circularly-profiled graft with an associated fixation device within an essentially “ovoid”-profiled vessel. Migration/dislocation also caused due to the use of attachment means which fasten only to the insubstantial, structurally compromised (diseased) intima and media levels of the vessel wall. Discontinuities of endoluminal profile are potential contributors to hemodynamic disturbances that might lead to non laminar, or even turbulent flow regimen. This in turn can contribute to increased clot formation. Thrombogenicity: manufactured of synthetic polymers, contemporary vascular liners present a luminal surface that is typically far more thrombogenic than the native arterial intimal tissue such devices cover

### SUMMARY OF THE INVENTION

One object of the invention is to disclose an extra-vascular wrapping (EVW) comprising a (i) wrapping adapted to at least partially encircle at least a segment of an aorta, especially an aneurysmatic aorta, and (ii) anchoring means located at the distal portion of said wrapping adapted to immobilize said wrapping to the aorto-iliac bifurcation via engagement of the aorto-iliac junction, located at the distal end of the wrapping, wherein said bifurcation-anchored wrapping is applied external to the aorta and further wherein the wrapping is collapse-resistant in the proximal-to-distal direction of the aorta.

Another object is to disclose an EVW as defined above, wherein the EVM wraps the aorta in a non-continuous manner.

Another object is to disclose an EVW as defined above, wherein the EVM additionally comprising a plurality of fastening means, adapted to definitively (i.e., effectively and reversibly) secure said wrapping over said aneurysmatic aorta.

Another object is to disclose an EVW as defined above, wherein the fastening means are selected from a group consisting of threads, screws, hooks, zips, fasteners, clips, flaps, claspers, springs, claspers, staplers, grips, zippers, hooks and corresponding eyes, hook and loop reclosable fastener squares, hook and loop reclosable fastener strips, hook and loop reclosable fastener dots, hooks-and-loops, e.g., Velcro™-type fasteners, straps, holes and string, wires, cables, tabs, poppers, nails, buttons and corresponding button holes, press buttons brackets, glues, adhesives, or any combination thereof.

Another object is to disclose an EVW as defined above, wherein the wrapping comprising at least one internal macroporous layer and at least one external microporous layer.

Another object is to disclose an EVW as defined above, wherein the internal macroporous layer, positioned directly over the aneurysmal aorta comprising voids dimensioned to allow free tissue ingrowth therethrough.

Another object is to disclose an EVW as defined above, wherein the internal macroporous layer is characterized by a polymer mesh with pores ranges from about 100 um to about 2 mm.

Another object is to disclose an EVW as defined above, wherein the external microporous layer, is made of a smoothly surfaced material, designed to prevent scar tissue formation and attachment thereto and thereby prevent internal organs from sticking thereto.

Another object is to disclose an EVW as defined above, wherein the external microporous layer is sparsely attached to said Internal macroporous layer, so as to allow said free tissue ingrowth while preventing scar tissue formation and attachment to said microporous layer.

Another object is to disclose an EVW as defined above, wherein at least one of said internal macroporous layer and external microporous layer comprising attaching means, said means are selected from a group consisting of threads, holes, screws, hooks, zips, fasteners, clips, flaps, claspers, springs, claspers, grips, zippers, hooks-and-loops e.g., Velcro™-type fasteners, straps, strings, wires, cables, tabs, poppers, nails, buttons, brackets, glue, adhesives, or any combination thereof.

Another object is to disclose an EVW as defined above, wherein the anchoring means is a gusset-like anchor adapted to be immobilized to the aorto-iliac junction aorto-iliac bifurcation.

Another object is to disclose an EVW as defined above, wherein the gusset-like anchor is interconnected to said wrapping by means of one or more connecting means.

Another object is to disclose an EVW as defined above, wherein the wrapping is characterized by a non-continuous bifurcated gusset-like structure comprising a main wrapping externally enveloping the aneurysmatic aorta, and at least one anchoring means, externally enveloping at least one iliac artery.

Another object is to disclose an EVW as defined above, wherein the wrapping is characterized by a non-continuous bifurcated pants-like structure comprising a main wrapping externally enveloping the aneurysmatic aorta, and at least one anchoring means, externally enveloping at least one iliac artery.

Another object is to disclose an EVW as defined above, wherein the at least one layer of said wrapping is wounded in two or more directions (e.g., in longitudinal and lateral directions, helically etc).

Another object is to disclose an EVW as defined above, wherein the EVW is characterized by initial folded configuration and by final expanded configuration; said folded configuration is adapted to be introduced and placed through a laparoscopic channel; said expanded configuration is adapted to encircle said aorta.

Another object is to disclose an EVW as defined above, wherein at least one portion or segment of said wrapping is having an interrupted cross-section over the aorta, such that said wrapping encircled the aneurysmatic aorta along a portion pC of said aorta's circumference.

Another object is to disclose an EVW as defined above, wherein the EVW additionally comprising a sustained drug delivery mechanism (SDDM).

Another object is to disclose a supported extra-vascular wrapping (SEVW) comprising (i) a wrapping adapted to at least partially encircle at least a segment of an aneurysmatic aorta, said wrapping having proximal and distal portions, said distal portion located adjacent the aorto-iliac bifurcation, (ii) a supporting frame, provided in at least on segment, layer of portion of said wrapping, adapted to resist collapse of said EVW in the proximal-to-distal direction and (iii) anchoring means in connection with both said the distal portion of said wrapping and said supporting frame, adapted to immobilize said wrapping to the aorto-iliac bifurcation via engagement of the aorto-iliac junction.

Another object is to disclose a ring-immobilized extra-vascular wrapping (REVW) comprising a wrapping adapted to at least partially encircle at least a segment of an aorta, especially an aneurysmatic aorta, and (ii) one or more ring-like anchoring means located at the distal portion of said wrapping adapted to immobilize said wrapping one or two iliac arteries.

Another object is to disclose a method for treating aneurysmatic aorta in an open-surgery procedure, comprising steps of: (a) identifying a subject having a medical condition indicative of an unacceptably high risk of rupture of an aneurysm of the abdominal aorta; (b) obtaining an extra-vascular wrapping (EVW) comprising a (i) wrapping adapted to at least partially encircle at least a segment of an aneurysmatic aorta, and (ii) anchoring means adapted to immobilize said wrapping to the aorto-iliac bifurcation via engagement of the aorto-iliac junction, wherein said EVM is wraps the aorta in a non-continuous manner; (c) surgically exposing said abdominal aorta; (d) positioning said EVM adjacent to the abdominal aorta of the subject; aligning a portion of fabric defining the proximal channel parallel to abdominal aorta and aligning portions of fabric defining a first, and possibly a second, distal channels parallel to iliac arteries; (e) wrapping the wrapping around the aorta, and anchoring said anchoring means to the one or two adjacent iliac arteries; and (f) connecting corresponding fastening means to one another.

Another object is to disclose a method for treating aneurysmatic aorta, this method is especially adapted for laparoscopic methodology, comprising steps of: (a) identifying a subject having a medical condition indicative of an unacceptably high risk of rupture of an aneurysm of the abdominal aorta; (b) obtaining an extra-vascular wrapping (EVW) comprising a (i) wrapping adapted to at least partially encircle at least a segment of an aneurysmatic aorta, and (ii) anchoring means adapted to immobilize said wrapping to the aorto-iliac bifurcation via engagement of the aorto-iliac junction, wherein said EVM is wraps the aorta in a non-continuous manner; (c) introducing a folded EVW through an endoscopic working channel to an abdominal location adjacent to said abdominal aorta of said subject; aligning a portion of wrapping defining a proximal channel parallel to abdominal aorta and aligning portions of said anchoring means defining a first, and optionally a second, distal channels parallel to iliac arteries; (d) endoscopically wrapping said wrapping around said aorta, (e) anchoring said anchoring means to the one or two adjacent iliac arteries; and, 0 endoscopically connecting corresponding fastening means to one another.

Another object is to disclose a method for treating an aneurysm of an abdominal aorta characterized by both (i) an internal intimal layer and an external adventitial layer, and (ii) bifurcates to two iliac arteries at an aorto-iliac junction; said method comprising steps of: (a) identifying a subject having a medical condition indicative of an unacceptably high risk of rupture of said aneurysm; (b) positioning a macroporous medical textile externally adjacent to the aorta for a period of time longer than about 2 weeks; wherein said textile comprising voids dimensioned to allow free ingrowth of the adventitial layer therethrough, and wherein said textile externally overlaps most of an external surface area of the aneurysm.

Still another object is to disclose a method as defined above, wherein said medical textile is immobilized over the aorta via engagement of the aorto-iliac junction.

Lastly, another object is to disclose a method as defined above, wherein the method additionally comprising selecting said EVW from a group consisting of pants-like structured EVW, gusset-like structured EVW, SDDM-EVW, supported EVW and ringed EVW.

### BRIEF EXPLANATIONS OF THE FIGURES

The present invention will now be described, by way of example only, with respect to the accompanying Figures, wherein:

FIG. 1 depicts the segment of the aorta below the diaphragm and the two bifurcating iliac arteries;

FIGS. 2 and 3 depict the folded and expanded (unfolded) configurations, respectively, of a multi-port extra-vascular support device with hooks and eyes realizing the fastening means;

FIGS. 4 and 5 depict the folded and expanded configurations, respectively, an extra-vascular bifurcated support device having holes that are adapted for a string to realize fastening of device;

FIGS. 6 and 7 depict the folded and expanded configurations, respectively, of an extra-vascular bifurcated support device having holes that are adapted for a string to realize fastening of device;

FIG. 8 depicts the folded configuration of an extra-vascular bifurcated support device having holes that are adapted for a string to realize fastening of device;

FIGS. 9 and 10 depict the folded and expanded configurations, respectively, of an extra-vascular bifurcated support device comprising a helically arranged support structure member, containing multiple turns about an abdominal aorta as a common longitudinal axis, and a fabric member spanning said support structure member, and hooks and eyes as fastening means, adapted to facilitate reduction of relative movement between adjacent turns of said support structure member;

FIGS. 11, 12 and 13 depict different variants of extra-vascular support devices in their expanded configurations, wherein solid gray stripes denote locations for distributing one type of hooks and loops reclosable fastener stripes (e.g. hooks) and hatched stripes denote the complementary (e.g. loops) type of hooks and loops reclosable fastener stripes;

FIG. 14(a) to (d) depict different types of fastening elements, taken as a realization example, for the textile industry;

FIG. 15 illustrated an SEVW according to one embodiment of the invention;

FIG. 16 illustrated an EVW according to another embodiment of the invention with recesses for the renal arteries; and,

FIG. 17, illustrating a ring-immobilized extra-vascular wrapping with a supporting frame (REVW-F, 140) according to yet another embodiment of the invention.

### DETAILED DESCRIPTION OF THE PREFERRED EMBODIMENTS

The description herein provided is means to provide the reader with exemplary embodiments of the present invention and the methods for their usage. The description is, however, not meant to be limiting in any way, to the spirit and scope of the invention, as set out in the appended claims.

A novel extra-vascular wrapping (EVW) is disclosed. The EVW comprising (i) at least one wrapping, characterized by a generally circular cross-section, adapted to at least partially encircle at least a segment of an aneurysmatic aorta; and (ii), anchoring means adapted to immobilize said wrapping to the aorto-iliac bifurcation via engagement of the aorto-iliac junction. The EVM wraps the aorta in a non-continuous manner. According to one embodiment of the invention, the non-consecutiveness is provided between (i) the warping and (ii), the anchoring means.

It is in the scope of the invention wherein the aforesaid wrapping is one integrated sleeve-like member. Alternatively, said wrapping is a segmented sleeve-like member comprising a plurality of interconnected segments, wherein the size, type and composition of each segment may differ. Hence for example, external segments may be more flexible than interior segments; one or more segments may comprise sustain drug delivery means; one or more segments may comprise anchoring means etc. The segmentation is provided longitudinally, i.e., along the aorta length; laterally and/or radially.

It is further in the scope of the invention wherein the aforesaid EVW comprises integrated or segmented wrapping; the wrapping comprises of at least one internal macroporous layer and at least one external macroporous layer. The Internal macroporous layer, positioned directly over the aneurysmal aorta, (incl. iliac arteries, etc.) may comprise pores. The pores may be localized in a determined array or provided in a dispersed manner. The pores may be in any shape or size, such as in the shape of holes, weaved apertures, netting and so forth. The size of the pores is determined in a manner that it would allow free tissue ingrowth into the pores.

The internal macroporous layer is possibly characterized by a polymer mesh with pores ranges from about 100 um to about 0.5 mm. The term ‘about’ refers hereinafter to any measure being ±25% of the determined value. This layer is designed to undergo quick incorporation into the adventitia, i.e. the external layer of the blood vessel, over which the wrapping is directly placed.

The external microporous layer is made of inert materials (e.g., either organic biocompatible non-sticking and non-adhesive, Teflon™-like materials, or inert inorganic Metalin™-like materials), designed to prevent internal organs from irreversibly attaching to said Internal macroporous layer. The external microporous layer is preferably sparsely attached to the internal macroporous layer or layers.

As disclosed above, at least one of the internal macroporous layer and the external microporous layer may comprise one or more attaching means, adapted to secure the in situ sleeve-like configuration of the external wrapping, while enveloping the aorta. The attaching means are selected in a non-limited manner from a group consisting of threads, screws, hooks, zips, fasteners, clips, flaps, claspers, springs, claspers, staplers, grips, zippers, hooks and corresponding eyes, hook and loop reclosable fastener squares, hook and loop reclosable fastener strips, hook and loop reclosable fastener dots, hooks-and-loops, e.g., Velcro™-type fasteners, straps, holes and string, wires, cables, tabs, poppers, nails, buttons and corresponding button holes, press buttons brackets, glue, adhesives, or any combination thereof.

At least a portion of the wrapping and/or anchoring means is manufactured in a non-limiting manner using fibers and materials having a linear mass density in the range between about 1 to about 100 denier. In a fabric-made wrapping and/or anchoring means, the fabric is made by means selected from a group of knitting, netting, weaving or braiding filaments of biocompatible matrices, e.g., interwoven fibers, mono-filaments, multi-filaments, yarns, membranes, films, sheets, tubes, e.g., polyester fiber, nylon fibers, polylactic (PLA) or polyglutamic (PGA) acids or other poly (alpha-hydroxy acid), polycaprolactone, polydioxanone, polygluconate, polylactic acid-polytheylene oxide copolymers, modified cellulose, collagen, poly(hydroxybutyrate), polyanhydride, polyphosphoester, poly(amino acids), polytheylene terephthalate (PET), expanded polytetra-fluorotheylene (ePTFE), polycarbonate urtheane (PCU), polyurtheane (PU) or a combination thereof. The fibers and materials preferably having imageable markers, e.g., markers distributed along the fastening means.

It is still in the scope of the invention wherein at least one layer of said wrapping is wounded in two or more common directions; such that adequate burst resistance (i.e. strength) is provided in all directions—radial, longitudinal, etc.

According to one embodiment of the invention, the aforesaid anchoring means is a gusset-like anchor, adapted to be immobilized to the aorto-iliac junction aorto-iliac bifurcation. This gusset-like anchor may be interconnected to the wrapping by means of one or more connecting means. In one possible manner, the connecting means are not encircling the full circumference of the aorto-arteries. Additionally or alternatively, the connecting means is one or more externally-attachable rings placed over each of the iliac arteries.

According to a similar embodiment of the invention, the aforesaid anchoring means are one or more ring-like members, adapted to encircle one or more iliac artery and thus to immobilize the wrapping. This type of externally applied anchoring means is made of materials selected inter alia from a group consisting of polymers, biodegradable, e.g., PLA, and non-degradable, e.g., PMMA, metal and metal alloys, e.g., stainless steel compositions, composite materials etc. Additionally or alternatively, those one or more ring-like members, adapted to encircle the aorta thus to immobilize the wrapping, are disclosed. Still another embodiment of the invention is an EVW as defined above, additionally comprising a supporting frame. The frame, made e.g., an elongated thermoplastic semi-rigid sheet-like polymeric member, is in connection with one of the rings and/or with the distal portion of the wrapping adjacent to said ring, end exceed along the aorta up to the wrapping's distal portion. By immobilizing the supporting frame to the distal portion of the wrapping, the collapse of the wrapping in the longitudinal direction of the aorta, especially in the distal end and adjacent to the aneurism bulge, is avoided.

According to yet another embodiment of the invention, the EVW is characterized by a non-continuous bifurcated pants-like structure (110), comprising a main wrapping, externally enveloping the aneurysmatic aorta, and at least one anchoring means, externally enveloping at least one iliac artery.

The Pants-like EVM may be characterized by a proximal portion comprising a proximal channel enveloping the aneurysmatic aorta, having a generally circular cross section, and a distal portion, comprising a first distal channel and an optional second distal channel having generally circular cross sections, the first and second distal channels having smaller diameters than the proximal channel, each is adapted to envelop one iliac artery. The proximal and the distal portions is having a first and a second distal channel lengths, respectively.

It is in the scope of the invention wherein length of proximal channel is ranges between about 5 to about 30 centimeter; wherein length of first and/or second distal channel ranges between about 1 to about 20 centimeters; wherein diameter of proximal channel ranges between about 2.5 to about 6 centimeters; wherein diameter of first and/or second distal channel ranges between about 1 to about 3 centimeters; and wherein thickness roughly ranges between about 0.05 to about 0.5 millimeters.

According to yet another embodiment of the invention, the EVW as defined in any of the above, is characterized by folded and expanded (i.e., unfolded and spread) configurations. The folded configuration is configured, e.g., (i) as a spiral wound useful to be reversibly accommodated within the inner lumen of inserting means, such as trocar, laparoscope, endoscopic working channel or other means having an internal diameter smaller than e.g., about 2 centimeters; (ii) as a tightly compressed patch, manually operated and expanded by a surgeon in a open-surgery; (iii) initially deflated EVM, adapted to be inflated, e.g., by commercially available ballooning means, to its final expanded configuration etc.

It is in the scope of the invention wherein the expanded configuration is secured by one or more fastening means, e.g., distributed on contour of either sides of said wrapping.

According to one embodiment of the invention, wherein at least one portion or segment of the wrapping as defined in any of the above is having a generally uninterrupted cross-section over its entire length. Hence, the wrapping encircled the aneurysmatic aorta along its entire circumference.

According to yet another embodiment of the invention, provided in addition to the above mention embodiment, or provided as an alternative, at least one portion or segment of the wrapping as defined in any of the above is having an interrupted cross-section over the aorta, such that the wrapping is characterized by a cross-section of less than 360°

An EVW with at least one portion or segment of a non-complete encirclement of the aorta is advantageous, e.g. for patients in which a portion of the aorta has fused with posterior ligaments of the spine. In some cases, a portion of the wrapping provides a complete (360° or more) encirclement of the aorta, e.g. just above the bifurcation.

Hence, the wrapping encircled the aneurysmatic aorta along only a portion pC of the circumference. The measure of said portion pC is varied, for example, from about 51% to about 95% of the aorta's external circumference. It is acknowledged in this respect that by additionally immobilizing at least a portion or segment of the wrapping to the aorta, e.g., by gluing mean etc, pC may be smaller than 50%.

It is also in the scope of the invention wherein an EVM with a sustained drug delivery mechanism (SDDM) is provided. The SDDM is obtained, e.g., by doping, immersing, coating, layering, socking, wetting, covalently or ionically binding, or otherwise attaching a predetermined medicament (one or more) to the inner layer, outer layer and/or central layer or a segment of the wrapping and.or anchoring means. It is in the scope of the invention wherein the medicament to be released is selected from a group consisting inter alia pharmaceuticals, tetracycline inhibitors, COX-2 inhibitors, ACE inhibitors, statins, steroids, non steroidal anti-inflammatory drugs, biocides, anti-coagulation agents or any combination thereof. In one embodiment, at least 70% of the medicament elutes from its carrier over a period in the range between about one to about 6-12 months

It is further in the scope of the invention wherein an EVW with at least one enforcement, supporting structure, skeleton and/or skeleton-like frame is disclosed. All term defined above will interchangeably referred in the text in the terms ‘frame’ and ‘support frame’).

The frame is adapted to provide the EVW with two characteristics, namely (i) to provide a stable wrapping which does not collapse along the longitudinal axis of the aorta; and (ii), to continuously and effectively attach the inner macroporous fabric layer to the outer surface of the diseased aorta, and thereby to enable incorporation of the fabric into the external surface (the adventitia layer) of the aorta, in a manner that further dilatation of the aneurismal aorta is prevented. Hence, the frame is adapted to resist collapsing of the EVW in the proximal-to-distal direction along the aorta's main longitudinal axis.

It is in the scope of the invention wherein the frame is provided with at least one segment, layer of portion of the EVM. Alternatively, the frame is provided along most of the length of the wrapping. According to one embodiment of the invention, the frame is an elastic support, made of, e.g., super-elastic metals such as cobalt chromium alloys; nickel-titanium alloys; electro-active polymers; polymers; composite materials or a combination thereof.

According to yet another embodiment of the invention, a set of new methods for treating aneurysmatic aorta is disclosed.

Hence, a first method is provided in an open-surgery procedure, and comprising the following steps is presented: (i) identifying a subject having a medical condition indicative of an unacceptably high risk of rupture of an aneurysm of the abdominal aorta; (ii) surgically exposing said abdominal aorta; (iii) positioning EVM as defined in any of the above, adjacent to the abdominal aorta of the subject, wherein portion of fabric defining the proximal channel is generally aligned parallel to abdominal aorta and portions of fabric defining said first and second distal channels are generally aligned parallel to the expected course of non diseased iliac arteries; (iv) wrapping the wrapping around the aorta, and anchoring said anchoring means to the one or two adjacent iliac arteries; and (v) connecting corresponding fastening means to one another such that a secured EVM is obtained.

A second method is provided in a laparoscopic methodology, comprising steps of (i) identifying a subject having a medical condition indicative of an unacceptably high risk of rupture of an aneurysm of the abdominal aorta; (ii) introducing a folded EVW through an endoscopic working channel to an abdominal location adjacent to said abdominal aorta of said subject, wherein portion of wrapping defining the proximal channel is generally aligned parallel to abdominal aorta and portions of the anchoring means defining the first and optionally the second distal channels are generally aligned parallel to the expected course of non diseased iliac arteries; (iii) endoscopically wrapping said wrapping around said aorta, and anchoring said anchoring means to the one or two adjacent iliac arteries; and, (iv) endoscopically connecting corresponding fastening means to one another.

A third method hereby disclosed is provided for treating an aneurysm of an abdominal aorta, particularly an aorta which comprises an internal intimal layer and an external adventitial layer; the aorta further bifurcates to two iliac arteries at an aorto-iliac junction. This method comprising steps of: (a) identifying a subject having a medical condition indicative of an unacceptably high risk of rupture of said aneurysm; (b) positioning a macroporous medical textile externally adjacent to the aorta for a period of time longer than about 2 weeks; wherein the textile comprising voids dimensioned to allow free ingrowth of the adventitial layer therethrough, and wherein the textile externally overlaps most (e.g., 75 percents or more) of an external surface area of the aneurysm. According to another embodiment of the invention, the medical textile is immobilized over the aorta via engagement of the aorto-iliac junction.

The term ‘medical textile’ in the context of the current invention preferably relates to any woven, non-woven, knitted, monofilament, or braided fabric, made from various biocompatible and mostly biostable commercially available polymers, such as viscose, lyocell, polyester, polypropylene, polyurethane, PTFE and ePTFE, polyamide, alginate, chitosan, silk, polyamide, elastomeric-fibres in general.

The term ‘macroporous medical textile’ in the context of the current invention preferably relates to a medical textile or any other material containing voids characterized by a typical dimension greater than about 50 micrometer. In a biologically-functional perspective, a ‘macroporous medical textile’ in the context of the current invention related to a fabric that would typically allow ingrowth of viable fibrous tissue therethrough. The term ‘microspores medical textile’ in the context of the current invention relates to a medical textile or any other material that is structured in a manner that would not allow the ingrowth of biological tissue therethrough. For example, an ePTFE film is a well known example of a biostable, implantable medical textile having such a property.

The term ‘proximal’ unless otherwise provided, relates to a location in the vasculature that is closer to the heart. The tern ‘distal’ unless otherwise provided, relates to a location in the vasculature that is farther away from the heart.

Those methods are similarly applied with any of the above mentioned EVWs, especially, yet not exclusively, with the aforesaid gasket-like, aforesaid pants-like EVW, supported EVW and ringed EVW.

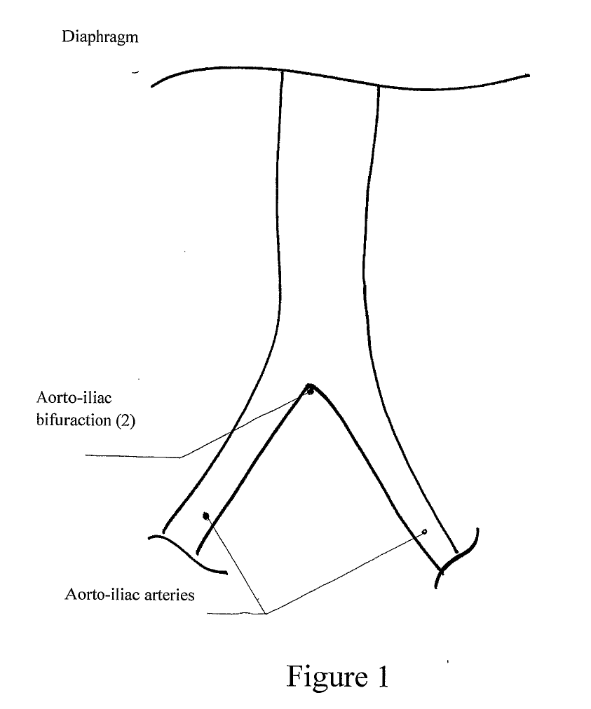

Reference is now made to FIG. 1 which illustrated in an out-of-scale manner a segment of the aorta below the diaphragm and the two bifurcating iliac arteries.

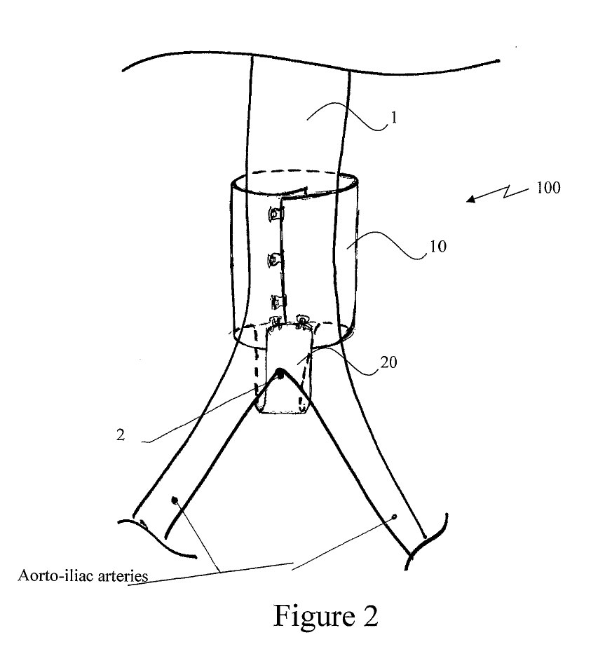

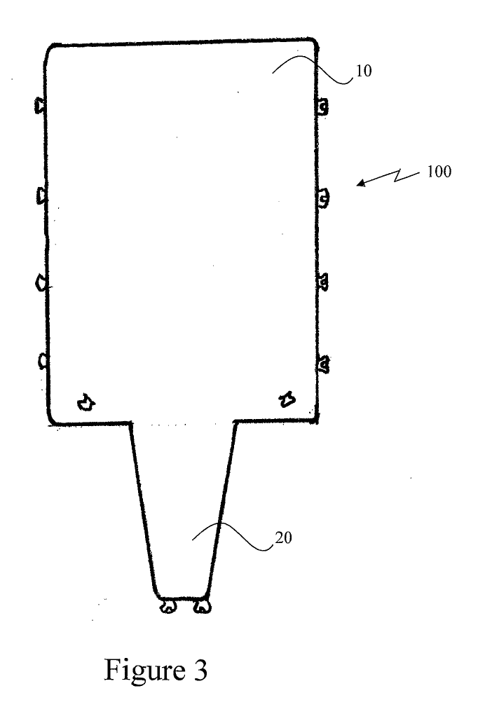

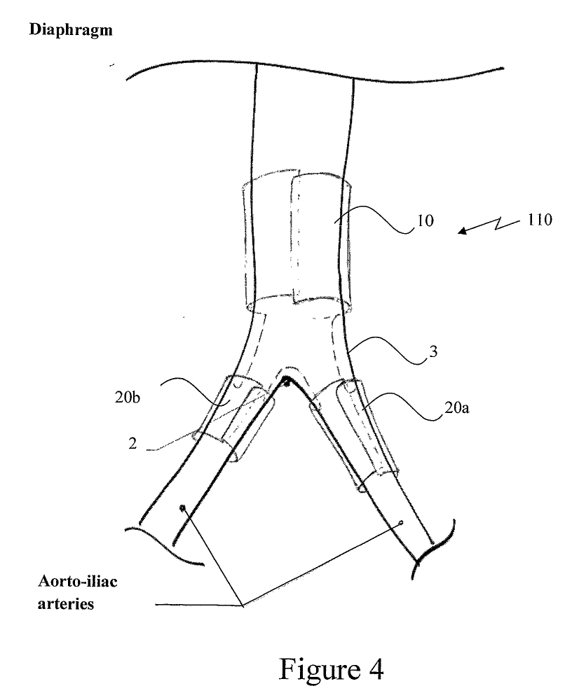

Reference is now made to FIGS. 2 and 3 which illustrate in an out-of-scale manner a gusset-like EVW (100) expanded and folded configurations, respectively. The gusset-like EVW (100) comprising a (i) wrapping (10) adapted to encircle a segment of the aneurysmatic aorta (1), and (ii) a single anchoring means (20) adapted to immobilize said wrapping to the aorto-iliac bifurcation via engagement of the aorto-iliac junction (2), wherein the EVM is wraps the aorta in a non-continuous manner: Sections 3a and 3b of the aorta are not enveloped by wrapping (10) nor by anchoring means (20).

Reference is now made to FIGS. 2 and 3 which illustrate in an out-of-scale manner a pants-like EVW (110) expanded and folded configurations, respectively. The pants-like EVW (110) comprising a (i) wrapping (10) adapted to encircle a segment of the aneurysmatic aorta (1), and (ii) a set of two anchoring means (20a, 20b) enveloping the iliac arteries, adapted to immobilize said wrapping to the aorto-iliac bifurcation via engagement of the aorto-iliac junction (2), wherein the EVM is wraps the aorta in a non-continuous manner: Section 3 of the aorta is not enveloped by wrapping (10) nor by the two anchoring means (20). The EEW is having a plurality of holes (40) that are adapted for a string to realize fastening of the wrapping and anchoring means.

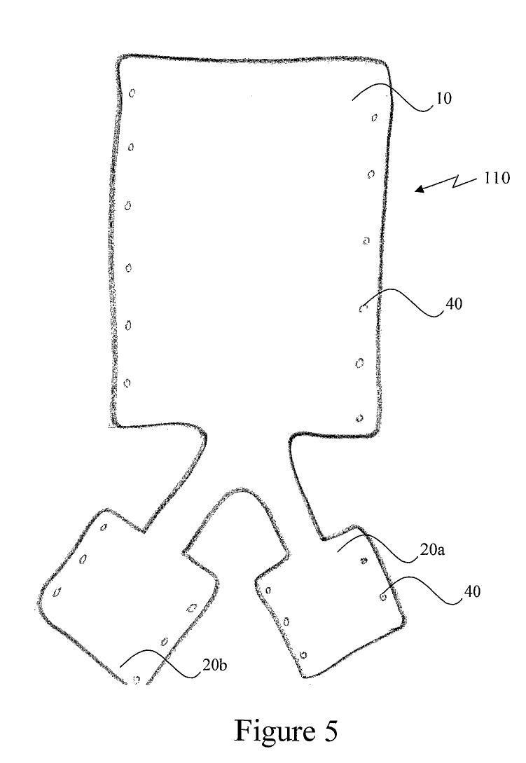

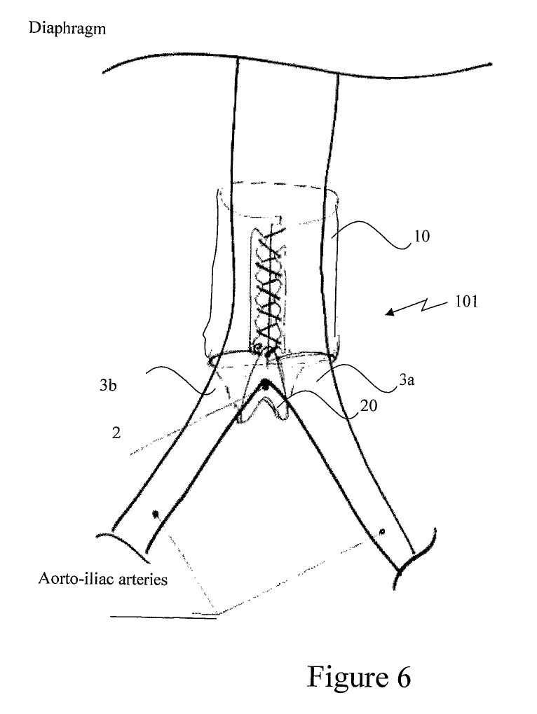

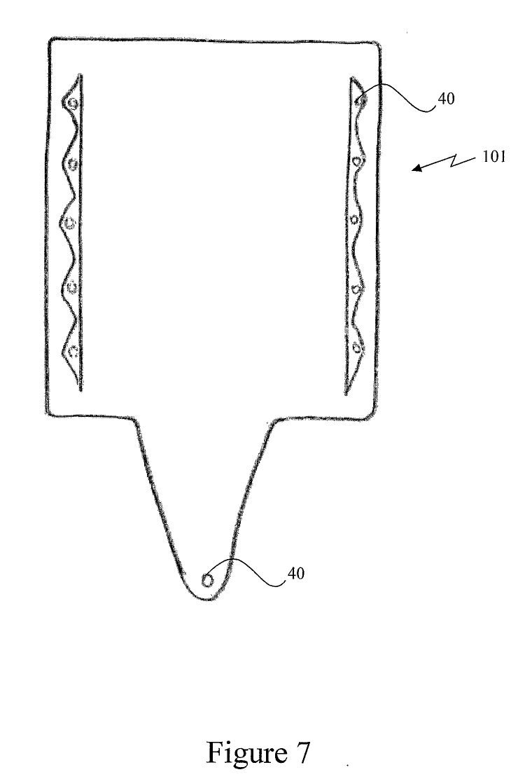

Reference is now made to FIGS. 6 and 5 which illustrate in an out-of-scale manner a gusset-like EVW (101) expanded and folded configurations, respectively. The gusset-like EVW (101) comprising a (i) wrapping (10) adapted to encircle a segment of the aneurysmatic aorta (1), and (ii) a single anchoring means (20) adapted to immobilize said wrapping to the aorto-iliac bifurcation via engagement of the aorto-iliac junction (2), wherein the EVM is wraps the aorta in a non-continuous manner: Sections 3a and 3b of the aorta are not enveloped by wrapping (10) nor by anchoring means (20). This EVW comprises a plurality of holes adapted for a string to realize fastening of the wrapping and anchoring means.

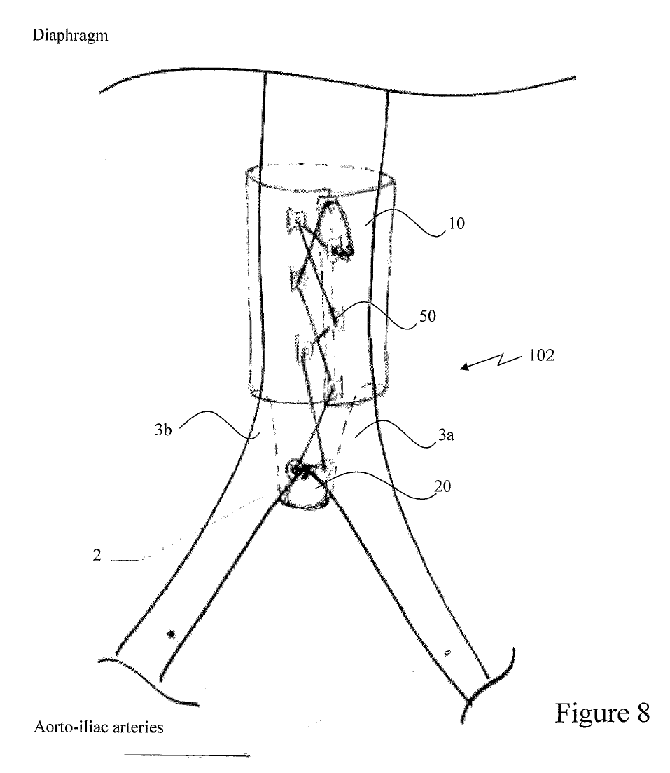

Reference is now made to FIG. 8 which illustrates in an out-of-scale manner a gusset-like EVW (102). The gusset-like EVW (102) comprising a (i) wrapping (10) adapted to encircle a segment of the aneurysmatic aorta (1), and (ii) a single anchoring means (20) adapted to immobilize said wrapping to the aorto-iliac bifurcation via engagement of the aorto-iliac junction (2), wherein the EVM is wraps the aorta in a non-continuous manner: Sections 3a and 3b of the aorta are not enveloped by wrapping (10) nor by anchoring means (20). The EVW is having a shoelace fastening mechanism (50).

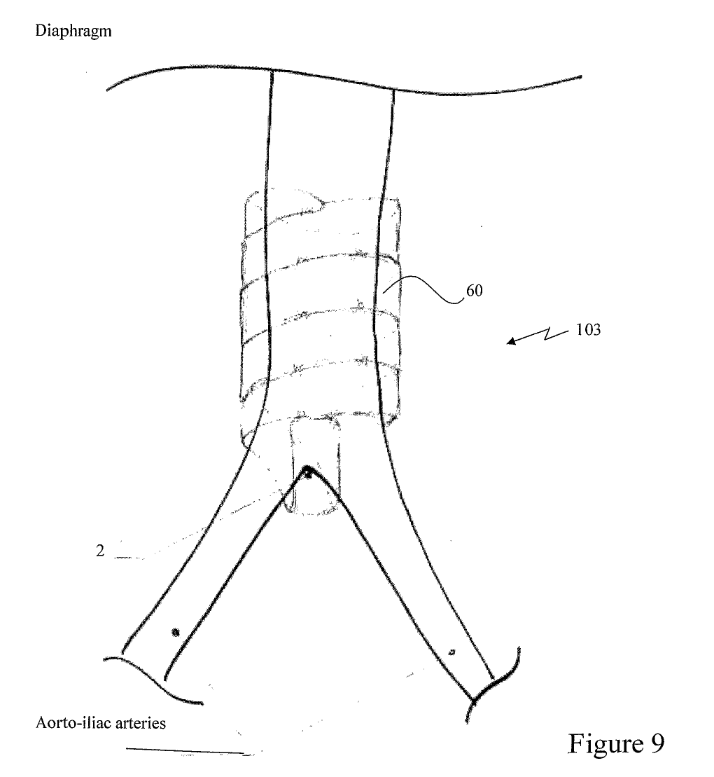

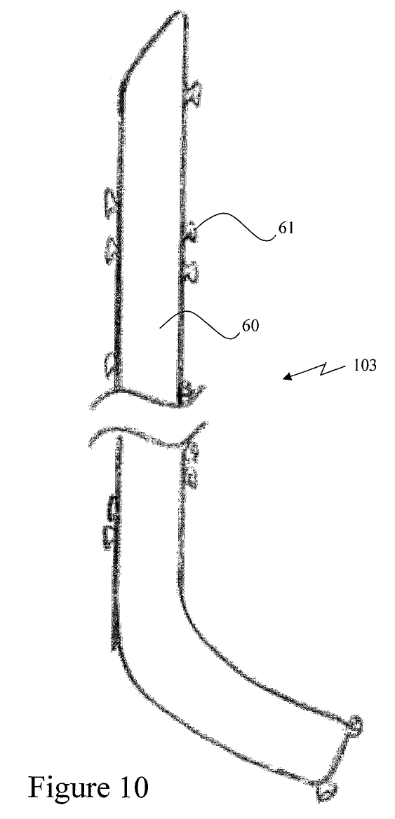

FIGS. 9 and 10 depict the final configuration and folded initial configurations of a gusset-like EVW (103), respectively. This type of EVM comprises of at least one helically arranged support structure member (60), containing multiple turns about an abdominal aorta as a common longitudinal axis, and a wrapping means, here made as a fabric member, spanning said support structure member, and hooks and eyes as fastening means (61), adapted to facilitate reduction of relative movement between adjacent turns of said support structure member.

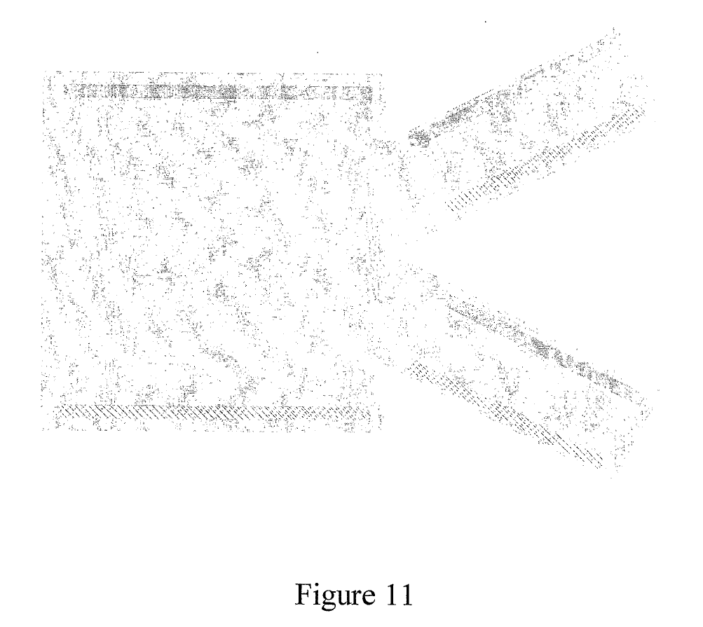

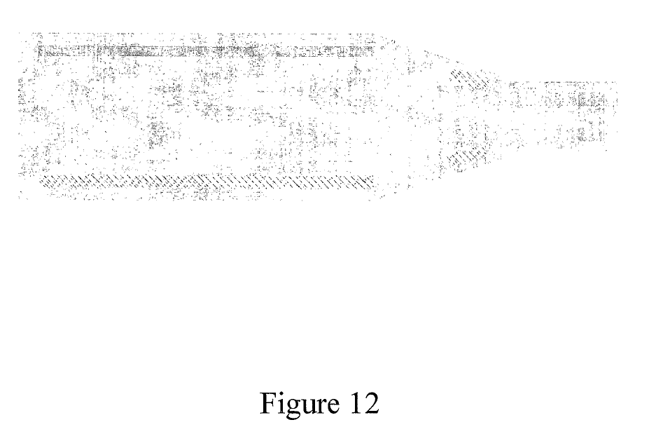

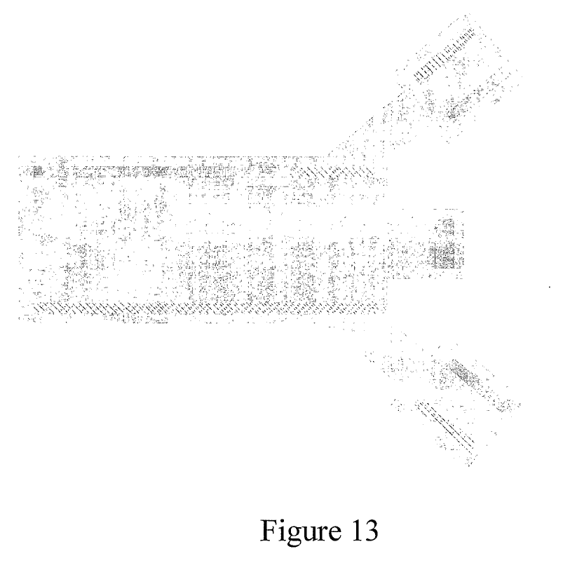

Reference is now made to FIGS. 11, 12 and 13 that depict in a out-of-scale manner different variants of extra-vascular support devices in their expanded state, wherein solid gray stripes denote locations for distributing one type of hooks and loops reclosable fastener stripes (e.g. hooks) and hatched stripes denote the complementary (e.g. loops) type of hooks and loops reclosable fastener stripes.

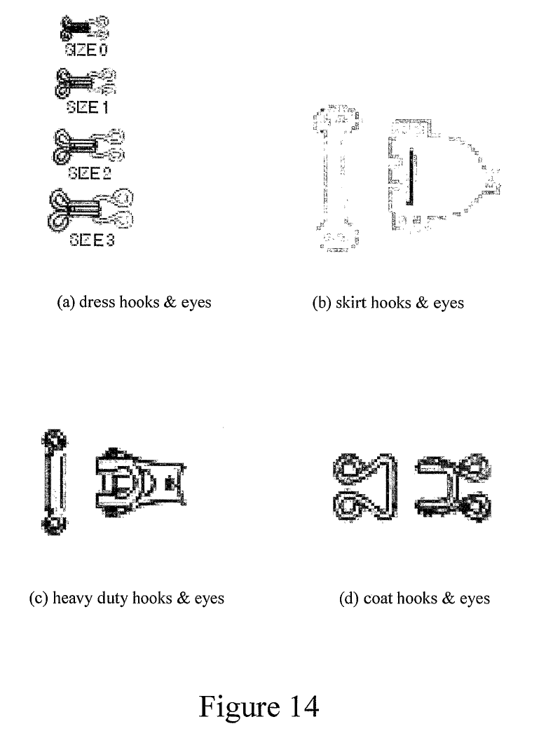

Reference is now made to FIGS. 14(a) and 14b that depict different types of fastening elements, taken as a realization example, for the textile industry.

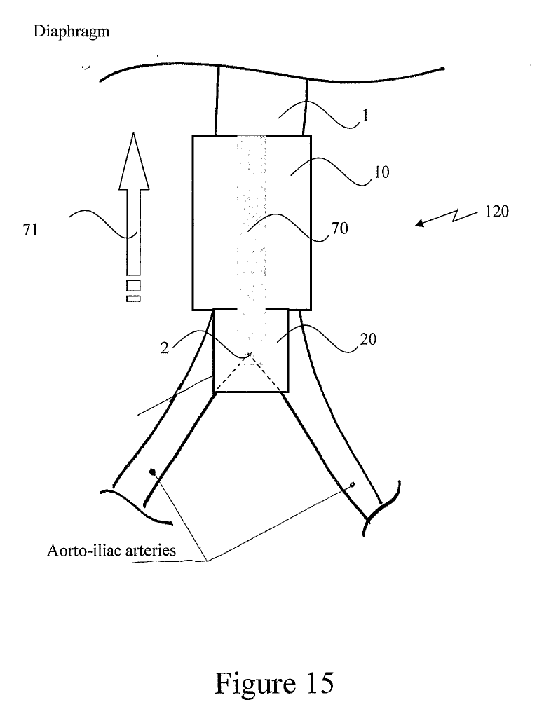

Reference is now made to FIG. 15, illustrating in a non-scaled manner a supported extra-vascular wrapping (SEVW, 120) according to one embodiment of the invention. This gusset-like SEVW (120) comprises of (z) one or more wrappings (here one, 10) adapted to at least partially encircle at least a segment of an aneurysmatic aorta (1), (ii) a supporting frame (70), provided in at least on segment, layer of portion of said warping, adapted to resist collapse of SEVW (120) in the proximal-to-distal direction (71) and (iii) anchoring means (20) in connection with both said wrapping and said supporting frame, adapted to immobilize said wrapping to the aorto-iliac bifurcation via engagement of the aorto-iliac junction.

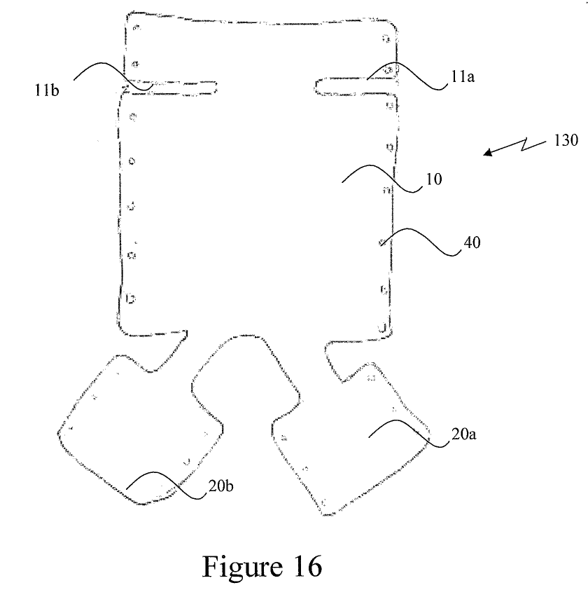

Reference is now made to FIG. 16, illustrating an out-of-scale scheme of a pants like EVW (130) comprising a wrapping (10) secured by sutures in holes (40), and two anchoring members (20a and 20b). The EVW 130 comprising in its spread configuration two recesses, a right renal artery recess (11a), and a left renal artery recess (11b).

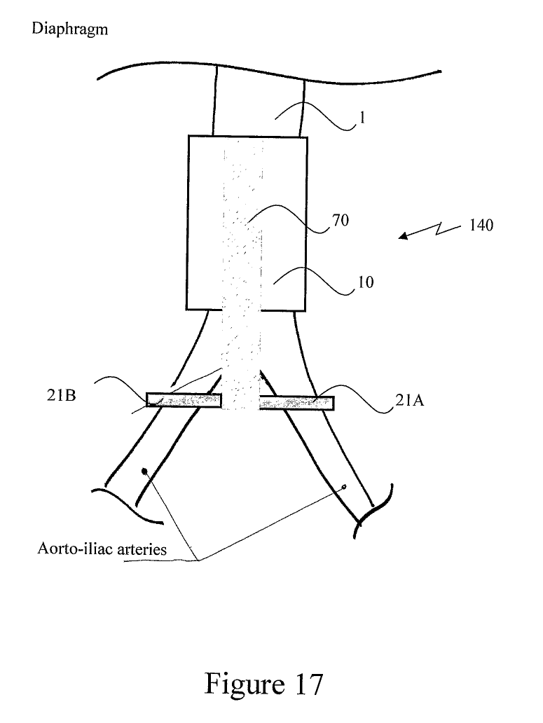

Lastly, reference is now made to FIG. 17, illustrating an out-of-scale scheme of a ring-immobilized extra-vascular wrapping with a supporting frame (REVW-F, 140) comprising a (i) wrapping (10) adapted to at least partially encircle at least a segment of an aorta (1), especially an aneurysmatic aorta, and (ii) one or more ring-like anchoring means (here two rings, 21A and 21B) located at the distal portion of said wrapping (10) adapted to immobilize said wrapping two iliac arteries. According to this specific embodiment, rings are attached to the wrapping by means of frame (70).

1. An extra-vascular wrapping (EVW) comprising a (i) wrapping adapted to at least partially encircle at least a segment of an aorta, especially an aneurysmatic aorta, and (ii) anchoring means located at the distal portion of said wrapping adapted to immobilize said wrapping to the aorto-iliac bifurcation via engagement of the aorto-iliac junction, wherein said bifurcation-anchored wrapping is applied external to the aorta and further wherein the wrapping is collapse-resistant in the proximal-to-distal direction of the aorta.

2. The EVW of claim 1, wherein said EVM wraps the aorta in a non-continuous manner.

3. The EVW of claim 1, additionally comprising a plurality of fastening means, adapted to secure said wrapping over said aneurysmatic aorta.

4. The EVW of claim 1, wherein said fastening means are selected from a group consisting of threads, screws, hooks, zips, fasteners, clips, flaps, claspers, springs, claspers, staplers, grips, zippers, hooks and corresponding eyes, hook and loop reclosable fastener squares, hook and loop reclosable fastener strips, hook and loop reclosable fastener dots, hooks-and-loops, e.g., Velcro™-type fasteners, straps, holes and string, wires, cables, tabs, poppers, nails, buttons and corresponding button holes, press buttons brackets, glues, adhesives, or any combination thereof.

5. The EVW of claim 1, wherein said wrapping comprising at least one internal macroporous layer and at least one external microporous layer.

6. The EVW of claim 5, wherein said internal macroporous layer, positioned directly over the aneurysmal aorta comprising voids dimensioned to allow free tissue ingrowth therethrough.

7. The EVW of claim 5, wherein said internal macroporous layer is characterized by a polymer mesh with pores ranges from about 100 μm to about 2 mm.

8. The EVW of claim 5, wherein said external microporous layer, is made of a smoothly surfaced material, designed to prevent scar tissue formation and attachment thereto and thereby prevent internal organs from sticking thereto.

9. The EVW of claim 5, wherein said external microporous layer is sparsely attached to said Internal macroporous layer, so as to allow said free tissue ingrowth while preventing scar tissue formation and attachment to said microporous layer

10. The EVW of claim 5, wherein at least one of said internal macroporous layer and external microporous layer comprising attaching means, said means are selected from a group consisting of threads, holes, screws, hooks, zips, fasteners, clips, flaps, claspers, springs, claspers, grips, zippers, hooks-and-loops e.g., Velcro™-type fasteners, straps, strings, wires, cables, tabs, poppers, nails, buttons, brackets, glue, adhesives, or any combination thereof.

11. The EVW of claim 1, wherein said anchoring means is a gusset-like anchor adapted to be immobilized to the aorto-iliac junction aorto-iliac bifurcation.

12. The EVW of claim 11, wherein said gusset-like anchor is interconnected to said wrapping by means of one or more connecting means.

13. The EVW of claim 11, wherein said wrapping is characterized by a non-continuous bifurcated gusset-like structure comprising a main wrapping externally enveloping the aneurysmatic aorta, and at least one anchoring means, externally enveloping at least one iliac artery.

14. The EVW of claim 1, wherein said wrapping is characterized by a non-continuous bifurcated pants-like structure comprising a main wrapping externally enveloping the aneurysmatic aorta, and at least one anchoring means, externally enveloping at least one iliac artery.

15. The EVW of claim 1, wherein at least one layer of said wrapping is wounded in two or more directions (e.g., in longitudinal and lateral directions, helically etc).

16. The EVW of claim 1, characterized by initial folded configuration and by final expanded configuration; said folded configuration is adapted to be introduced and placed through a laparoscopic channel; said expanded configuration is adapted to encircle said aorta.

17. The EVW of claim 1, wherein at least one portion or segment of said wrapping is having an interrupted cross-section over the aorta, such that said wrapping encircled the aneurysmatic aorta along a portion pC of said aorta's circumference.

18. The EVW of claim 1, additionally comprising a sustained drug delivery mechanism (SDDM).

19. A supported extra-vascular wrapping (SEVW) comprising (i) at wrapping adapted to at least partially encircle at least a segment of an aneurysmatic aorta, said wrapping having proximal and distal portions, said distal portion located adjacent the aorto-iliac bifurcation, (ii) a supporting frame, provided in at least on segment, layer of portion of said wrapping, adapted to resist collapse of said EVW in the proximal-to-distal direction and (iii) anchoring means in connection with both said the distal portion of said wrapping and said supporting frame, adapted to immobilize said wrapping to the aorto-iliac bifurcation via engagement of the aorto-iliac junction.

20. A ring-immobilized extra-vascular wrapping (REVW) comprising a (i) wrapping adapted to at least partially encircle at least a segment of an aorta, especially an aneurysmatic aorta, and (ii) one or more ring-like anchoring means located at the distal portion of said wrapping adapted to immobilize said wrapping one or two iliac arteries.

21. A method for treating aneurysmatic aorta in an open-surgery procedure, comprising steps of: (a) identifying a subject having a medical condition indicative of an unacceptably high risk of rupture of an aneurysm of the abdominal aorta; (b) obtaining an extra-vascular wrapping (EVW) comprising a (i) wrapping adapted to at least partially encircle at least a segment of an aneurysmatic aorta, and (ii) anchoring means adapted to immobilize said wrapping to the aorto-iliac bifurcation via engagement of the aorto-iliac junction, wherein said EVM is wraps the aorta in a non-continuous manner; (c) surgically exposing said abdominal aorta; (d) positioning said EVM adjacent to the abdominal aorta of the subject; aligning a portion of fabric defining the proximal channel parallel to abdominal aorta and aligning portions of fabric defining a first, and possibly a second, distal channels parallel to iliac arteries; (e) wrapping the wrapping around the aorta, and anchoring said anchoring means to the one or two adjacent iliac arteries; and (f) connecting corresponding fastening means to one another.

22. The method for treating aneurysmatic aorta according to claim 21, adapted to laparoscopic methodology, comprising steps of: (a) identifying a subject having a medical condition indicative of an unacceptably high risk of rupture of an aneurysm of the abdominal aorta; (b) obtaining an extra-vascular wrapping (EVW) comprising a (i) wrapping adapted to at least partially encircle at least a segment of an aneurysmatic aorta, and (ii) anchoring means adapted to immobilize said wrapping to the aorto-iliac bifurcation via engagement of the aorto-iliac junction, wherein said EVM is wraps the aorta in a non-continuous manner; (c) introducing a folded EVW through an endoscopic working channel to an abdominal location adjacent to said abdominal aorta of said subject; aligning a portion of wrapping defining a proximal channel parallel to abdominal aorta and aligning portions of said anchoring means defining a first, and optionally a second, distal channels parallel to iliac arteries; (d) endoscopically wrapping said wrapping around said aorta, (e) anchoring said anchoring means to the one or two adjacent iliac arteries; and, (f) endoscopically connecting corresponding fastening means to one another.

23. A method for treating an aneurysm of an abdominal aorta characterized by both (i) an internal intimal layer and an external adventitial layer, and (ii) bifurcates to two iliac arteries at an aorto-iliac junction; said method comprising steps of: (a) identifying a subject having a medical condition indicative of an unacceptably high risk of rupture of said aneurysm; (b) positioning a macroporous medical textile externally adjacent to the aorta for a period of time longer than about 2 weeks; wherein said textile comprising voids dimensioned to allow free ingrowth of the adventitial layer therethrough, and wherein said textile externally overlaps most of an external surface area of the aneurysm.

24. The method of claim 23, wherein said medical textile is immobilized over the aorta via engagement of the aorto-iliac junction.

25. The method according to claim 21, additionally comprising selecting said EVW from a group consisting of pants-like structured EVW, gusset-like structured EVW, SDDM-EVW, supported EVW and ringed EVW.

26. The method according to claim 23, additionally comprising selecting said EVW from a group consisting of pants-like structured EVW, gusset-like structured EVW, SDDM-EVW, supported EVW and ringed EVW.
